SEQUENCE LISTING (2 sequences)

SEQ ID NO: 1 - protein, 211 aa, Homo sapiens
  SIGNAL (1)..(19)
  PROPEP (20)..(75)
  mat_peptide (78)..(211)
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 2 - DNA, 4171 bp, Homo sapiens
agcctgcgctcctggcgccctcatgtcttcacgggactccccgcgccggttgacgtggtgtctcgttcggatctccaggcaagacctcagctccggcagcagcatcagacaaaccagtctcgtgctcccaggcagtgcgcccagaggaggcgcagagcgcggcagctgccgctgagccgcccgcagcgccccgggcccgcgcagccccagccaagagcgcgacgcgcgcagccctgtcagccccccacccaaagcagcggcggctgctcggacctcggcttctgggggtgcgggggcccggcgggagagttgccggcagccctcgccctgttggcggcggcggcggcgggagtcttggccgccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

(19) United States
(12) Patent Application Publication
(10) Pub. No.: US 2024/0366723 A1
(43) Pub. Date: Nov. 7, 2024

(54) COMBINATION THERAPY FOR THE TREATMENT OF ENTERIC NEUROPATHIES

(71) Applicant: TRANSFERT PLUS, LIMITED PARTNERSHIP, Quebec (CA)

(72) Inventors: Nicolas PILON, Montréal (CA); Rodolphe SORET, Montréal (CA)

(21) Appl. No.: 18/567,915

(22) PCT Filed: Jun. 15, 2022
(86) PCT No.: PCT/CA2022/050955

§ 371 Date: Dec. 7, 2023

Related U.S. Application Data
(60) Provisional application No. 63/202,549, filed on Jun. 16, 2021.

(51) Int. Cl. A61K 38/18; A61K 31/19; A61P 1/00; A61P 25/02

(52) U.S. Cl. CPC A61K 38/185; A61K 31/19; A61P 1/00; A61P 25/02

(57) ABSTRACT

The present application relates to a method for treating a human subject suffering from an enteric neuropathy such as Hirschsprung disease (HSCR) or intestinal hypoganglionosis through the administration of an effective dose of a combination comprising a Glial cell line-Derived Neurotrophic Factor (GDNF) polypeptide and a short chain fatty acid such as butyric acid.

### CROSS REFERENCE TO RELATED APPLICATIONS

The present application claims the benefit of U.S. provisional patent application No. 63/202,549 filed on Jun. 16, 2021, which is incorporated herein by reference.

### TECHNICAL FIELD

The present disclosure generally relates to the treatment of enteric neuropathies such as Hirschsprung disease (HSCR) and intestinal hypoganglionosis.

### BACKGROUND ART

The enteric nervous system (ENS) extends along the entire gastrointestinal tract to control bowel motility, blood flow and epithelial activity in response to sensory stimuli (1). Interconnected enteric ganglia containing neurons and glia develop from neural crest-derived progenitors that migrate through the intestine during prenatal development. Incomplete colonization of distal colon by ENS progenitors causes Hirschsprung disease (HSCR), a condition affecting 1 in 5000 newborns (2,3). In HSCR, distal colon without neural ganglia (i.e., aganglionic colon) remains tonically contracted and does not propagate contractions, causing functional intestinal obstruction. HSCR symptoms include refractory constipation with retention of stool and air, abdominal distension, growth failure, occasional vomiting, bowel inflammation (enterocolitis) and a risk of bacterial translocation into blood causing sepsis and premature death (2).

HSCR is clinically subdivided into short-segment (S-HSCR) and long-segment forms (L-HSCR) (4). S-HSCR, which occurs in >80% of cases, means the ENS is absent from rectum and sigmoid colon. L-HSCR means longer regions of distal bowel are aganglionic. HSCR etiology remains incompletely understood, but many genes influence HSCR risk (2). Furthermore, genetic risk variants may combine with non-genetic factors to prevent full bowel colonization by ENS progenitors (5). This non-Mendelian inheritance occurs because many proteins must work together for normal ENS development.

Since 1948, most children with HSCR have had their life saved by surgical removal of distal aganglionic bowel (16, 17). However, this procedure is far from ideal. Post-surgical complications are common and can also be long-lasting, impacting survival (e.g., enterocolitis) and/or quality of life (e.g., fecal incontinence or obstructive symptoms) (18-20). One promising approach would be “regenerative medicine” to rebuild the ENS and reduce the need for surgery. This idea prompted many groups to develop cell transplantation-based HSCR therapies (21). However, despite many encouraging results, some difficulties remain (22). The optimal source of stem cells, ideal amplification and/or differentiation strategies prior to transplantation, methods of cell delivery, and cell function and fate after transplantation are not yet well defined. Additionally, non-autonomous cell transplantation may require immunosuppression.

Hypoganglionosis, also known as intestinal hypoganglionosis, is a disorder causing a reduced number of nerves in the intestinal wall. Intestinal hypoganglionosis can mimic HSCR; patients with both conditions may present with chronic constipation, intestinal obstruction, and enterocolitis (inflammation of the intestines). Patients with hypoganglionosis may also suffer from severe complications including fecaloma (hardening of the feces inside the colon), bleeding or perforation of the intestine, and breathing problems resulting from a distended colon. The exact cause of hypoganglionosis is often not known. In some cases, it is due to factors present at birth (congenital), while other times it is believed to be an acquired condition. The management of isolated hypoganglionosis generally involves surgery to remove the affected bowel segment.

There is thus clearly a need for alternative treatments for enteric neuropathies such as HSCR and intestinal hypoganglionosis, notably treatments aimed at inducing neurogenesis in the distal colon and restoring distal colon motility in HSCR and intestinal hypoganglionosis patients.

The present description refers to a number of documents, the content of which is herein incorporated by reference in their entirety.

### SUMMARY OF THE INVENTION

The present disclosure relates to the use of a combination of GDNF and a short-chain fatty acid for the treatment of one or more pathological features of enteric neuropathies such as Hirschsprung disease.

In aspects and embodiment, the present disclosure relates to the following items 1 to 50:1.

1. A method for treating a human subject suffering from an enteric neuropathy, the method comprising administrating to the subject an effective amount of (i) a Glial cell line-Derived Neurotrophic Factor (GDNF) polypeptide; and (ii) a short chain fatty acid (SCFA), or a pharmaceutically acceptable salt or ester thereof.
2. The method of item 1, wherein the GDNF polypeptide comprises an amino acid sequence having at least 70% identity with amino acids 78-211 of SEQ ID NO:1 (FIG. 3A).
3. The method of item 2, wherein the GDNF polypeptide comprises an amino acid sequence having at least 90% identity with amino acids 78-211 of SEQ ID NO:1 (FIG. 3A).
4. The method of item 3, wherein the GDNF polypeptide comprises an amino acid sequence having at least 95% identity with amino acids 78-211 of SEQ ID NO:1 (FIG. 3A).

5. The method of item 4, wherein the GDNF polypeptide comprises amino acids 78-211 of SEQ ID NO:1 (FIG. 3A).

6. The method of any one of items 1 to 5, wherein the effective amount of GDNF polypeptide administered to the human subject corresponds to a dose of about 5 μg to about 20 μg in a mouse pup.
7. The method of any one of items 1 to 6, wherein the SCFA is a C3-C5 SCFA.
8. The method of any one of items 1 to 7, wherein the SCFA is butyric acid.
9. The method of any one of items 1 to 8, wherein the GDNF polypeptide and/or the SCFA are present in a pharmaceutical composition that further comprises a pharmaceutically acceptable carrier.
10. The method of item 9, wherein the pharmaceutically acceptable carrier comprises a saline solution or a gelling agent.
11. The method of any one of items 1 to 10, wherein the GDNF polypeptide and/or the SCFA are administered rectally through enema.
12. The method of any one of items 1 to 10, wherein the GDNF polypeptide and/or the SCFA are administered by injection into the distal colon wall.
13. The method of any one of items 1 to 12, wherein the GDNF polypeptide and/or the SCFA are administered once-a-day up to four times a day.
14. The method of any one of items 1 to 13, wherein the GDNF polypeptide and/or the SCFA are administered for at least 2 consecutive days.
15. The method of any one of items 1 to 14, wherein the method is performed prior to surgical removal of the aganglionic or hypoganglionic segment in the subject.
16. The method of any one of items 1 to 15, wherein the method is performed after surgical removal of the aganglionic or hypoganglionic segment in the subject.
17. The method of item 15 or 16, wherein the surgical removal of the aganglionic or hypoganglionic segment is through pull-through surgery.
18. The method of any one of items 1 to 17, wherein the enteric neuropathy is intestinal hypoganglionosis.
19. The method of any one of items 1 to 17, wherein the enteric neuropathy is Hirschsprung disease (HSCR).
20. The method of item 19, wherein the subject suffers from short-segment HSCR.
21. The method of item 16 or 17, wherein the HSCR is Collagen VI-associated HSCR.
22. The method of any one of items 1 to 21, wherein the human subject is less than 5-year-old.
23. The method of item 22, wherein the human subject is less than 6-month-old.
24. The method of any one of items 1 to 23, wherein the GDNF polypeptide and/or the SCFA are administered into the rectum and/or the sigmoid colon.
25. The method of item 29, wherein the pharmaceutical composition is administered or is for administration into the rectosigmoid region.
26. A combination for use in treating a human subject suffering from an enteric neuropathy, the combination comprising (i) a GDNF polypeptide; and (ii) an SCFA, or a pharmaceutically acceptable salt or ester thereof.
27. The combination for use according to item 26, wherein the GDNF polypeptide comprises an amino acid sequence having at least 70% identity with amino acids 78-211 of SEQ ID NO:1 (FIG. 3A).
28. The combination for use according to item 27, wherein the GDNF polypeptide comprises an amino acid sequence having at least 90% identity with amino acids 78-211 of SEQ ID NO:1 (FIG. 3A).
29. The combination for use according to item 28, wherein the GDNF polypeptide comprises an amino acid sequence having at least 95% identity with amino acids 78-211 of SEQ ID NO:1 (FIG. 3A).
30. The combination for use according to item 29, wherein the GDNF polypeptide comprises amino acids 78-211 of SEQ ID NO:1 (FIG. 3A).
31. The combination for use according to any one of items 26 to 30, wherein the dose of recombinant GDNF polypeptide used corresponds to a dose of about 5 μg to about 20 μg in a mouse pup.
32. The combination for use according to any one of items 26 to 31, wherein the SCFA is a C3-C5 SCFA.
33. The combination for use according to any one of items 26 to 32, wherein the SCFA is butyric acid.
34. The combination for use according to any one of items 26 to 33, wherein the GDNF polypeptide and/or the SCFA are present in a pharmaceutical composition that further comprises a pharmaceutically acceptable carrier.
35. The combination for use according to item 34, wherein the pharmaceutically acceptable carrier comprises a saline solution or a gelling agent.

36. The combination for use according to any one of items 26 to 35, wherein the GDNF polypeptide and/or the SCFA are administered rectally through enema.

37. The combination for use according to any one of items 26 to 35, wherein the GDNF polypeptide and/or the SCFA are administered by injection into the distal colon wall.
38. The combination for use according to any one of items 26 to 37, wherein the GDNF polypeptide and/or the SCFA are administered once-a-day up to four times a day.
39. The combination for use according to any one of items 26 to 38, wherein the GDNF polypeptide and/or the SCFA are administered for at least 2 consecutive days.
40. The combination for use according to any one of items 26 to 39, wherein the combination is for use prior to surgical removal of the aganglionic or hypoganglionic segment in the subject.
41. The combination for use according to any one of items 26 to 40, wherein the combination is for use after surgical removal of the aganglionic or hypoganglionic segment in the subject.
42. The method of item 40 or 41, wherein the surgical removal of the aganglionic or hypoganglionic segment is through pull-through surgery.
43. The combination for use according to any one of items 26 to 42, wherein the enteric neuropathy is intestinal hypoganglionosis.
44. The combination for use according to any one of items 26 to 42, wherein the enteric neuropathy is Hirschsprung disease (HSCR).
45. The combination for use according to item 44, wherein the subject suffers from short-segment HSCR.
46. The combination for use according to item 44 or 45, wherein the HSCR is Collagen VI-associated HSCR.
47. The combination for use according to any one of items 26 to 46, wherein the human subject is less than 5-year-old.
48. The combination for use according to item 47, wherein the human subject is less than 6-month-old.
49. The combination for use according to any one of items 26 to 48, wherein the GDNF polypeptide and/or the SCFA are for administration into the rectum and/or the sigmoid colon.
50. The combination for use according to item 49, wherein the GDNF polypeptide and/or the SCFA are for administration into the rectosigmoid region.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A is a graph showing the survival of homozygous Holstein^{Tg/Tg} mice following administration of vehicle, butyrate (5 mM), GDNF (1 μg/μl) and a combination of butyrate+GDNF.

FIG. 1B depicts the statistical analysis of survival assay shown in FIG. 1A.

FIG. 1C depicts the survival rate per specific time points shown in FIG. 1A.

FIG. 2A is a graph showing the survival of homozygous Holstein^{Tg/Tg} mice following administration of GDNF alone or in combination with various neurotrophic factors.

FIG. 2B depicts the survival rate per specific time points shown in FIG. 2A.

FIG. 2C depicts the statistical analysis of survival assay shown in FIG. 2A.

FIG. 3A shows the amino acid sequence of human GDNF isoform 1 (UniProtKB accession No. P39905, SEQ ID NO:1), with the sequence corresponding to the signal peptide underlined (residues 1-19), the sequence corresponding to the propeptide italicized (residues 20-75) and the sequence corresponding to the mature polypeptide in bold (residues 78-211).

FIGS. 3B-C show the nucleotide sequence of the cDNA encoding human GDNF isoform 1 (RefSeq accession No. NM_000514.4, SEQ ID NO:2), with the sequence encoding the signal peptide underlined (nucleotides 562-618), the sequence encoding the propeptide italicized (nucleotides 619-786) and the sequence encoding the mature polypeptide in bold (nucleotides 793-1194).

### DISCLOSURE OF INVENTION

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein using a mouse model of enteric neuropathy (e.g., Hirschsprung disease (HSCR)), the present inventors show that administration of recombinant GDNF in combination with the short chain fatty acid (SCFA) butyrate in the distal colon significantly improve survival. While butyrate alone did not have any effect on survival, it was shown to significantly potentiate the effect of recombinant GDNF. These results provide evidence that recombinant GDNF in combination with short chain fatty acid may be used for the treatment of enteric neuropathies (e.g., ENS defects such as HSCR), i.e., for improving one or more of the pathological features of enteric neuropathies and/or survival, in human patients.

Accordingly, in a first aspect, the present disclosure provides a method for treating a human subject suffering from an enteric neuropathy (e.g., Hirschsprung disease (HSCR) or intestinal hypoganglionosis), the method comprising administrating to the subject an effective amount of (i) Glial cell line-Derived Neurotrophic Factor (GDNF) and (ii) a short chain fatty acid (SCFA), or a pharmaceutically acceptable salt or ester thereof. The present disclosure also provides the use of (i) GDNF and (ii) an SCFA, or a pharmaceutically acceptable salt or ester thereof, for treating a human subject suffering from an enteric neuropathy (e.g., Hirschsprung disease (HSCR) or intestinal hypoganglionosis). The present disclosure also provides the use of (i) GDNF and (ii) an SCFA, or a pharmaceutically acceptable salt or ester thereof, for the manufacture of a medicament for treating a human subject suffering from an enteric neuropathy (e.g., Hirschsprung disease (HSCR) or intestinal hypoganglionosis). The present disclosure also provides a combination for use in treating a human subject suffering from an enteric neuropathy (e.g., Hirschsprung disease (HSCR) or intestinal hypoganglionosis), the combination comprising (i) GDNF and (ii) an SCFA, or a pharmaceutically acceptable salt or ester thereof.

The present disclosure also provides a method for restoring distal colon motility and/or epithelial barrier in a human subject suffering from an enteric neuropathy (e.g., HSCR or intestinal hypoganglionosis), the method comprising administrating an effective amount of (i) Glial cell line-Derived Neurotrophic Factor (GDNF) and (ii) a short chain fatty acid (SCFA), or a pharmaceutically acceptable salt or ester thereof. The present disclosure also provides the use of an effective amount of (i) GDNF; and (ii) a short chain fatty acid (SCFA), or a pharmaceutically acceptable salt or ester thereof, for restoring distal colon motility in a human subject suffering from an enteric neuropathy (e.g., HSCR or intestinal hypoganglionosis). The present disclosure also provides the use of an effective amount of (i) GDNF; and (ii) a short chain fatty acid (SCFA), or a pharmaceutically acceptable salt or ester thereof for the manufacture of a medicament for restoring distal colon motility in a human subject suffering from an enteric neuropathy (e.g., HSCR or intestinal hypoganglionosis). The present disclosure also provides a combination for use in restoring distal colon motility in a human subject suffering from an enteric neuropathy (e.g., HSCR or intestinal hypoganglionosis), the combination comprising (i) GDNF and (ii) an SCFA, or a pharmaceutically acceptable salt or ester thereof.

The present disclosure also provides a method for inducing enteric neurogenesis in an aganglionic or hypoganglionic segment of the distal colon of a human subject suffering from an enteric neuropathy (e.g., HSCR or intestinal hypoganglionosis), the method comprising administering an effective amount of (i) GDNF; and (ii) a short chain fatty acid (SCFA), or a pharmaceutically acceptable salt or ester thereof. The present disclosure also provides the use of (i) GDNF; and (ii) a short chain fatty acid (SCFA), or a pharmaceutically acceptable salt or ester thereof, for inducing enteric neurogenesis in an aganglionic or hypoganglionic segment of the distal colon of a human subject suffering from an enteric neuropathy (e.g., HSCR or intestinal hypoganglionosis), wherein the composition is for administration into the distal colon of the subject. The present disclosure also provides the use of (i) GDNF; and (ii) a short chain fatty acid (SCFA), or a pharmaceutically acceptable salt or ester thereof, for the manufacture of a medicament for inducing enteric neurogenesis in an aganglionic or hypoganglionic segment of the distal colon of a human subject suffering from an enteric neuropathy (e.g., HSCR or intestinal hypoganglionosis). The present disclosure also provides a combination for use in inducing enteric neurogenesis in an aganglionic or hypoganglionic segment of the distal colon of a human subject suffering from an enteric neuropathy (e.g., HSCR or intestinal hypoganglionosis), the combination comprising (i) GDNF and (ii) an SCFA, or a pharmaceutically acceptable salt or ester thereof.

The term “short chain fatty acid” (SCFA) as used herein refers to a fatty acid of 2 to 5 carbon atoms, which includes acetic acid (C2), propionic acid (C3), butyric acid and isobutyric acid (C4), as well as valeric acid, isovaleric acid and 2-methylbutanoic acid (C5). Pharmaceutically acceptable salts of SCFAs such as pharmaceutically acceptable acetates, proprionates, butyrates, isobutyrates, valerates, isovalerates and 2-methylbutanoates may also be administered/used in the methods and uses described herein. As used herein, the term “pharmaceutically acceptable salt” is intended to mean those salts which retain the biological effectiveness and properties of the free acids, which are not biologically or otherwise undesirable. These salts are derived from addition of an inorganic base or an organic base to the organic acid. Such salts include alkali metal salts such as sodium, lithium, and potassium salts; alkaline earth metal salts such as calcium and magnesium salts; metal salts such as aluminium salts, iron salts, zinc salts, copper salts, nickel salts and a cobalt salts; inorganic amine salts such as ammonium or substituted ammonium salts, such as trimethylammonium salts; and salts with organic bases (for example, organic amines) such as chloroprocaine salts, dibenzylamine salts, dicyclohexylamine salts, dicyclohexylamines, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, tris-(2-hydroxyethyl)amine salts, tris(hydroxymethyl)aminomethane salts as well as amino acid salts such as L-ornithine salts, L-arginine salts, and L-lysine). Such salts can be formed quite readily by those skilled in the art using standard techniques. Indeed, the chemical modification of a pharmaceutical compound (i.e., drug) into a salt is a technique well known to pharmaceutical chemists, (See, e.g., H. Ansel et al., Pharmaceutical Dosage Forms and Drug Delivery Systems (6^{th} Ed. 1995) at pp. 196 and 1456-1457). Salts of SCFAs may be formed, for example, by reacting the SCFA with an amount of acid or base, such as an equivalent amount, in a medium such as one in which the salt precipitates or in an aqueous medium followed by lyophilization.

In an embodiment, the pharmaceutically acceptable salt of the SCFA is a metal salt, such as a sodium, potassium, lithium, ammonium, calcium or magnesium salt. In a further embodiment, the pharmaceutically acceptable salt of the SCFA is a sodium salt.

Esters of SCFAs may also be administered/used in the methods and uses of the present disclosure. Examples of SCFA esters are triglycerides of SCFAs that are composed of the SCFA (three molecules) and glycerol. Triglycerides of SCFAs are metabolized in vivo to release the three SCFAs molecules and are thus considered prodrugs of SCFAs. Examples of triglycerides of SCFAs include triacetin, triproprionin, tributyrin, triisobutyrin, trivalerin and triisovalerin.

The term “enteric neuropathy” as used herein refers to a disease associated with abnormalities in the ENS, including abnormal development of the ENS, e.g., abnormal number of neurons (hypoganglionosis, aganglionosis) and/or abnormal differentiation of neurons. Examples of enteric neuropathies include enteric dysganglionoses such as HSCR and intestinal hypoganglionosis. In an embodiment, the enteric neuropathy is HSCR. In another embodiment, the enteric neuropathy is intestinal hypoganglionosis.

The expression “inducing enteric neurogenesis” as used herein refers to an increase in the production of enteric neurons and/or enteric glial cells relative to prior to treatment with the composition comprising a human GDNF polypeptide. The enteric nervous system comprises various types of neurones including enteric primary afferent neurons (EPANs), excitatory circular muscle motorneurons, inhibitory circular muscle motorneurons, longitudinal muscle motorneurons, ascending interneurons, descending interneurons, secretomotor and vasomotor neurons, and intestinofugal neurons, as well as enteric glial cells (EGCs) that provide structural support to neurons and contribute to neuronal maintenance, survival, and function (Costa et al., Gut 2000; (Suppl IV) 47: iv15-iv19; De Giorgio et al., American Journal of Physiology-Gastrointestinal and Liver Physiology, Vol. 303, No. 8: G887-G893, 2012). The production of one or more of these cell types may be induced by the administration/use of the combination described herein.

In another embodiment, the administration/use of the combination described herein reduces the infiltration of inflammatory or immune cells (e.g., neutrophils) in the colon (e.g., distal colon). In another embodiment, the administration/use of the combination described herein restores (partly or completely) the proportions of immune cells in the colon (e.g., distal colon). In another embodiment, the administration/use of the combination described herein restores epithelial impermeability.

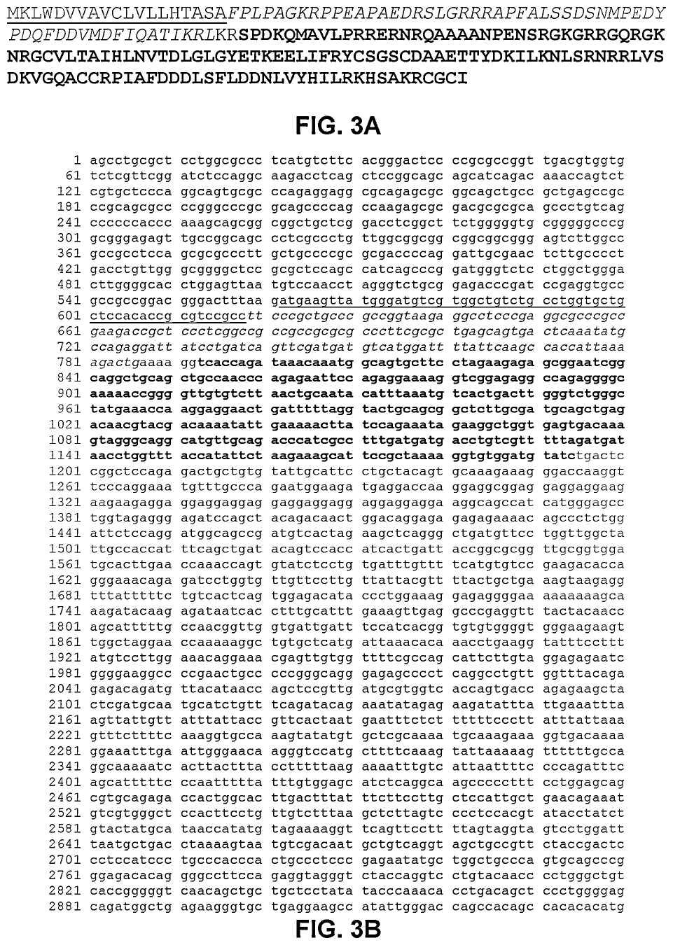

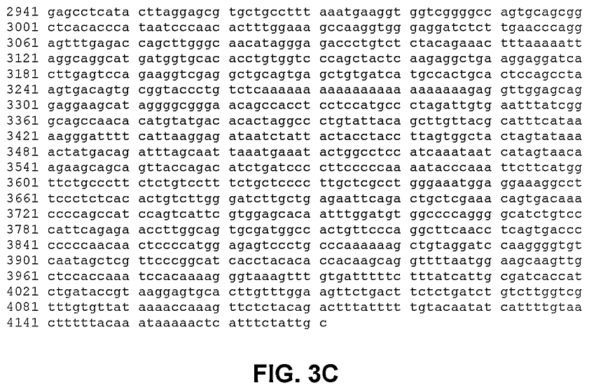

The term “human GDNF polypeptide” as used herein refers to the native mature human GDNF protein, or to functional variants or fragments thereof that retain a biological activity of the native mature human GDNF protein, e.g., the ability to bind to a GDNF receptor (particularly the “rearranged during transfection” (RET) proto-oncogene and/or the Neural Cell Adhesion Molecule (NCAM) receptor) and trigger a signal in a cell expressing a GDNF receptor (e.g., RET and/or NCAM). The amino acid sequence of native human GDNF protein (isoform 1, the canonical sequence) is depicted in FIG. 3A (SEQ ID NO:1), with the sequence corresponding to the mature protein (residues 78-211) highlighted in bold. The GDNF precursor protein is processed to a mature secreted form that exists as a homodimer. Each GDNF monomer contains seven conserved cysteine residues, including Cys-101, which is used for inter-chain disulfide bridging, and others that are involved in the intramolecular ring formation known as the cysteine-knot configuration.

In an embodiment, the human GDNF polypeptide is a recombinant human GDNF polypeptide. The term “recombinant” when made in reference to a protein or a polypeptide refers to a protein or polypeptide molecule that is not isolated from a natural source (e.g., biological sample), e.g., which is expressed from a recombinant nucleic acid construct created by means of molecular biological techniques. Referring to a nucleic acid construct as “recombinant” therefore indicates that the nucleic acid molecule has been manipulated using genetic engineering, i.e., by human intervention. Recombinant nucleic acid constructs may for example be introduced into a host cell by transformation (e.g., transduction or transfection).

Functional variants or fragments of native mature human GDNF protein may include one or more amino acid substitutions, deletions and/or additions relative to the native mature human GDNF protein, and may have a biological activity that is lower, equivalent or higher than that of the native mature human GDNF protein. In an embodiment, the functional variant or fragment has an activity that is equivalent (e.g., between 90% to 110%) or higher (e.g., more than 110%) to that of the native mature human GDNF protein. In an embodiment, the variant comprises one or more conservative substitutions. Conservative amino acid substitutions are known in the art, and include amino acid substitutions in which one amino acid having certain physical and or chemical properties is exchanged for another amino acid that has the same chemical or physical properties. For instance, the conservative amino acid substitution can be an acidic amino acid substituted for another acidic amino acid (e.g., Asp to Glu or vice-versa), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.). In another embodiment, the variants can comprise the amino acid sequence of the native GDNF protein or polypeptide with at least one non-conservative amino acid substitution. Preferably, the non-conservative amino acid substitution(s) enhance(s) the activity of the variant relative to that of the native mature human GDNF protein. In an embodiment, the human GDNF polypeptide has the ability to bind to the RET receptor. In an embodiment, the human GDNF polypeptide has the ability to bind to the NCAM receptor. In an embodiment, the human GDNF polypeptide has the ability to bind to the GDNF family co-receptor alpha (GFRalpha) 1-3.

In an embodiment, the human GDNF polypeptide comprises at least 10, 15 or 20 amino acids (e.g., contiguous amino acids) from the mature human native GDNF protein. In an embodiment, the human GDNF polypeptide comprises the sequence ETTYDKILKNLSRNR (gliafin), which corresponds to residues 153-167 of SEQ ID NO: 1 and is the putative binding domain of human GDNF to the NCAM receptor (see, Nielsen et al., J Neurosci. 2009 Sep. 9; 29 (36): 11360-11376). In other embodiments, the human GDNF polypeptide comprises at least 25, 30, 35, 40, 45, 50, 60, 70, 80, 90 or 100 amino acids (e.g., contiguous amino acids) from the mature human native GDNF protein, including residues 153-167 of SEQ ID NO: 1.

In an embodiment, the human GDNF polypeptide comprises an amino acid sequence that is at least 50%, 60% or 70% identical to the sequence of residues 78-211 depicted in FIG. 3A (SEQ ID NO:1). In another embodiment, the human GDNF polypeptide comprises an amino acid sequence that is at least 80% identical to the sequence of residues 78-211 depicted in FIG. 3A (SEQ ID NO:1). In another embodiment, the human GDNF polypeptide comprises an amino acid sequence that is at least 85% identical to the sequence of residues 78-211 depicted in FIG. 3A (SEQ ID NO:1). In another embodiment, the human GDNF polypeptide comprises an amino acid sequence that is at least 90% identical to the sequence of residues 78-211 depicted in FIG. 3A (SEQ ID NO:1). In another embodiment, the human GDNF polypeptide comprises an amino acid sequence that is at least 95% identical to the sequence of residues 78-211 depicted in FIG. 3A (SEQ ID NO:1). In another embodiment, the human GDNF polypeptide comprises an amino acid sequence that is at least 98% identical to the sequence of residues 78-211 depicted in FIG. 3A (SEQ ID NO:1). In another embodiment, the human GDNF polypeptide comprises an amino acid sequence that is at least 99% identical to the sequence of residues 78-211 depicted in FIG. 3A (SEQ ID NO:1). In another embodiment, the human GDNF polypeptide comprises or consists of the sequence of residues 78-211 depicted in FIG. 3A (SEQ ID NO:1). “Identity” refers to sequence identity between two polypeptides. Identity can be determined by comparing each position in the aligned sequences. Methods of determining percent identity are known in the art, and several tools and programs are available to align amino acid sequences and determine a percentage of identity including EMBOSS Needle, ClustalW, SIM, DIALIGN, etc. As used herein, a given percentage of identity with respect to a specified subject sequence, or a specified portion thereof, may be defined as the percentage of amino acids in the candidate derivative sequence identical with the amino acids in the subject sequence (or specified portion thereof), after aligning the sequences and introducing gaps, if necessary to achieve the maximum percent sequence identity, as generated by the Smith Waterman algorithm (Smith & Waterman, J. Mol. Biol. 147:195-7 (1981)) using the BLOSUM substitution matrices (Henikoff & Henikoff, Proc. Natl. Acad. Sci. USA 89:10915-9 (1992)) as similarity measures. A “% identity value” is determined by the number of matching identical amino acids divided by the sequence length for which the percent identity is being reported.

Covalent modifications of the human GDNF polypeptide are included within the scope of this disclosure. For example, the native glycosylation pattern of the human GDNF polypeptide may be modified (Beck et al., Curr. Pharm. Biotechnol. 9:482-501, 2008; Walsh, Drug Discov. Today 15:773-780, 2010), and linking the human GDNF polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. The human GDNF polypeptide may comprise one or more modifications that confer additional biological properties to the polypeptide such as protease resistance, plasma protein binding, increased plasma half-life, tissue or intracellular penetration, etc. Such modifications include, for example, covalent attachment of molecules/moiety to the polypeptide such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485).

The human GDNF polypeptide may also be conjugated to moieties to induce its multimerization or oligomerization (e.g., tetramerization), for example by fusing the human GDNF polypeptide to an oligomerization domain or to a molecule that may be oligomerized (e.g., biotin that may bind to 4 binding sites on streptavidin). The human GDNF polypeptide may also be conjugated to moieties that will target the GDNF polypeptide to the distal colon or to specific cells of the distal colon (e.g., Schwann cells and/or precursor thereof, enteric glial cells, pericytes), for example using an antibody, antibody fragment or ligand that binds to a marker present on cells from the distal colon.

The human GDNF polypeptide can also be conjugated to one or more therapeutic or active agents (e.g., to a drug, or to another polypeptide to form a fusion polypeptide). Any method known in the art for conjugating the human GDNF polypeptide to another moiety (e.g., active agent) may be employed, including those methods described by Hunter et al. (1962) Nature, 144:945; David et al. (1974) Biochemistry, 13:1014; Pain et al. (1981) J. Immunol. Meth., 40:219; Nygren, J. Histochem. and Cytochem., 30:407 (1982), and Hermanson, Bioconjugate Techniques 1996, Academic Press, Inc., San Diego. The human GDNF polypeptide may be conjugated to another moiety either directly or through a linker.

In an embodiment, the GDNF polypeptide and/or SCFA (or pharmaceutically acceptable salt or ester thereof) is formulated in a pharmaceutical composition. The GDNF polypeptide and SCFA (or pharmaceutically acceptable salt or ester thereof) may be formulated in the same pharmaceutical composition or in distinct pharmaceutical compositions. In an embodiment, the GDNF polypeptide and the SCFA (or pharmaceutically acceptable salt or ester thereof) are formulated in distinct pharmaceutical compositions. In an embodiment, the GDNF polypeptide and the SCFA (or pharmaceutically acceptable salt or ester thereof) are formulated in the same pharmaceutical composition. Such pharmaceutical compositions typically comprise one or more pharmaceutically acceptable excipients.

An “excipient” as used herein, has its normal meaning in the art and is any ingredient that is not an active ingredient (drug) itself. Excipients include for example buffers, binders, lubricants, diluents, fillers, thickening agents, disintegrants, plasticizers, coatings, barrier layer formulations, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present composition is not limited in these respects. The carrier/excipient can be suitable, for example, for intravenous, parenteral, subcutaneous, intramuscular, intracranial, intraorbital, ophthalmic, intraventricular, intracapsular, intraspinal, intrathecal, epidural, intracisternal, intraperitoneal, intranasal, rectal or pulmonary (e.g., aerosol) administration. Therapeutic compositions are prepared using standard methods known in the art by mixing the active ingredient having the desired degree of purity with one or more optional pharmaceutically acceptable carriers, excipients and/or stabilizers (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press).

In an embodiment, the GDNF polypeptide and/or SCFA (or pharmaceutically acceptable salt or ester thereof) is formulated for oral administration. Formulations suitable for oral administration may include (a) liquid solutions, such as an effective amount of active agent(s)/composition(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient(s), carriers known in the art.

In an embodiment, the GDNF polypeptide and/or SCFA (or pharmaceutically acceptable salt or ester thereof) is formulated for parenteral administration (e.g., injection). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylenevinyl acetate copolymer particles, osmotic pumps, implantable infusion systems, and liposomes. Formulations for inhalation may contain excipients, (e.g., lactose) or may be aqueous solutions containing, for example, polyoxyethylene-9-lauryl ether, glycocholate and deoxycholate, or may be oily solutions for administration in the form of nasal drops, or as a gel.

In an embodiment, the GDNF polypeptide and/or SCFA (or pharmaceutically acceptable salt or ester thereof) is formulated for enteric delivery, i.e., delivery into the intestines. This may be achieved by methods well known in the art. For example, the GDNF polypeptide and/or SCFA (or pharmaceutically acceptable salt or ester thereof) may be coated or encapsulated with an enteric agent or material. Enteric agents for instance allow release at certain pHs or in the presence of degradative enzymes or bacteria that are characteristically present in specific locations of the GI tract (e.g., small intestine, large intestine, or specific regions thereof) where release is desired. In an embodiment, the enteric material is pH-sensitive and is affected by changes in pH encountered within the gastrointestinal tract (pH-sensitive release). The enteric material typically remains insoluble at gastric pH, then allows for release of the active ingredient in the higher pH environment of the downstream gastrointestinal tract (e.g., often the duodenum, or sometimes the colon). In another embodiment, the enteric material comprises enzymatically degradable polymers that are degraded by bacterial enzymes (e.g., carbohydrate processing enzymes such as glycosidases, polysaccharide lyases and carbohydrate esterases) present in the lower gastrointestinal tract, particularly in the colon. Such enteric materials include, for example, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the trade-name Acryl-EZE® (Colorcon, USA), Eudragit® (Rohm Pharma; Westerstadt, Germany), including Eudragit® L30D-55 and L100-55 (soluble at pH 5.5 and above), Eudragit® L-IOO (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different enteric materials may also be used. Approaches for colon specific drug delivery are well known in the art (see, e.g., Philip et al., Oman Med J. 2010 April; 25 (2): 79-87; Lee et al., Pharmaceutics. 2020 January; 12 (1): 68), and include pH-dependent systems (e.g., using pH-dependent polymers), receptor-mediated systems, magnetically-driven systems, delayed or time-dependent systems, microbially triggered drug delivery systems (e.g., comprising sugar-based polymers that may be degraded by enzymes produced by the colon microflora such as glucoronidase, xylosidase, arabinosidase, galactosidase), pressure controlled colonic delivery capsule (drug release induced by the higher pressures encountered in the colon), osmotic controlled drug delivery, as well as any combinations of these approaches (e.g., colon targeted delivery system (CODESTM) using a combined approach of pH dependent and microbially triggered drug delivery).

In an embodiment, the GDNF polypeptide and/or SCFA (or pharmaceutically acceptable salt or ester thereof) is formulated in a capsule made of an enteric material (enteric capsule). In an embodiment, the GDNF polypeptide and/or SCFA (or pharmaceutically acceptable salt or ester thereof) is formulated for administration into the distal colon of the subject.

The term “distal colon” as used herein refers to the last three segments of the colon, namely the descending colon, the sigmoid colon and the rectum. In an embodiment, the pharmaceutical composition is administered or is for administration into the rectum and/or the sigmoid colon. In an embodiment, the pharmaceutical composition is administered or is for administration into the rectosigmoid region, which comprises the last part of the sigmoid colon and the beginning of the rectum. The skilled person would understand that the GDNF polypeptide and/or SCFA (or pharmaceutically acceptable salt or ester thereof) may be administered directly into the distal colon, or may be administered at a site away from the distal colon but using suitable means to provide delivery of the GDNF polypeptide and/or SCFA (or pharmaceutically acceptable salt or ester thereof) into the distal colon. For example, the formulation(s) may comprise a coating that is specifically degraded under the conditions (e.g., pH, enzymatic environment, bacterial environment, etc.) of the distal colon, and thus the formulation(s) may be administered in another region of the gastro-intestinal system but the GDNF polypeptide and/or SCFA (or pharmaceutically acceptable salt or ester thereof) will only be released once the formulation reaches the colon, and more specifically the distal colon. Approaches for colon specific drug delivery are well known in the art (see, e.g., Philip et al., Oman Med J. 2010 April; 25 (2): 79-87; Lee et al., Pharmaceutics. 2020 January; 12 (1): 68), and include pH-dependent systems (e.g., using pH-dependent polymers), receptor-mediated systems, magnetically-driven systems, delayed or time-dependent systems, microbially triggered drug delivery systems (e.g., comprising sugar-based polymers that may be degraded by enzymes produced by the colon microflora such as glucoronidase, xylosidase, arabinosidase, galactosidase), pressure controlled colonic delivery capsule (drug release induced by the higher pressures encountered in the colon), osmotic controlled drug delivery, as well as any combinations of these approaches (e.g., colon targeted delivery system (CODESTM) using a combined approach of pH dependent and microbially triggered drug delivery).

Formulations for rectal/distal colon administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the GDNF polypeptide and/or SCFA (or pharmaceutically acceptable salt or ester thereof). More recently, liquid suppositories have been developed. Liquid suppositories typically contain thermosensitive and/or mucoadhesive polymers such as poloxamers, Carbopol® (crosslinked polyacrylic acid polymers), sodium alginate, polycarbophil, hydroxypropyl methylcellulose (HPMC), hydroxyethyl cellulose, and methylcellulose.

Formulations for rectal/distal colon administration may also be presented as an enema, a liquid-drug solution, suspension or emulsion that is injected into the rectum and the distal colon. The liquid in which the GDNF polypeptide and/or SCFA (or pharmaceutically acceptable salt or ester thereof) is diluted may be water or a saline solution or suspension, for example.

Formulations for rectal/distal colon administration may also be in the form of a rectal foam or gel. Rectal gels are semi-solid formulations that contain a solvent trapped within a polymer network to create a viscous consistency. Viscosity of the gel can be modified by the addition of co-solvents (e.g., glycerin and propylene glycol) and electrolytes. Foams comprise a hydrophilic liquid continuous phase containing a foaming agent and a gaseous dispersion phase distributed throughout. Following rectal administration, they transition from a foam state to a liquid or semi-solid state on the mucosal surface. Foaming agents are typically amphiphilic substances that are important for foam generation and stabilization. The molecules contain hydrophilic components that are soluble in the aqueous phase and hydrophobic components that form micelles to minimize contact with the aqueous phase.

Administration into the rectum/distal colon may be performed using currently available endoscopes or specialized catheters designed for rectal administration or injection into the distal colon wall of medications and liquids, which may be placed safely and remain comfortably in the rectum for repeated use.

The GDNF polypeptide and/or SCFA (or pharmaceutically acceptable salt or ester thereof) may be administered according to any suitable dosage regimen, for example four times-a-day, twice-a-day, once-a-day, twice-a-week, once-a-week, etc. The treatment may be performed for any suitable period of time to achieve the desired effect, for example for 1 week, 2 weeks, 3 weeks or more.

In an embodiment, the effective dose of GDNF polypeptide administered or for administration to the human subject corresponds to a dose of about 5 μg to about 20 μg in a mouse pup, which is the range shown to be effective in the studies described herein. A 10 μl enema comprising a recombinant GDNF solution was administered to mouse pups. 10 μl is estimated to correspond to the volume necessary to fill the distal colon and rectum of the pups. Accordingly, administration of 5 μg GDNF in mice (pups) is achieved by administering 10 μl of a 0.5 μg/ul GDNF solution, and administration of 20 μg GDNF in mice (pups) is achieved by administering 10 μl of a 2.0 μg/ul GDNF solution. The volume required to fill the distal colon and rectum of a human baby may be estimated using the formula: 10 ml×weight of the baby (in kg). Accordingly, a dose of 5 μg GDNF in mice corresponds to about 5 mg per kg in a human baby, and a dose of 20 μg GDNF in mice corresponds to about 20 mg per kg in a human baby. Thus, in an embodiment, the effective dose of GDNF polypeptide administered or for administration to the human subject is about 5 mg to about 20 mg per kg, preferably about 10 mg to about 15 mg per kg. In an embodiment, the GDNF polypeptide is administered or is for administration through a 0.5 mg/ml to 2 mg/ml composition (solution or gel).

In an embodiment, the combination of therapeutic agents (GDNF polypeptide and SCFA or pharmaceutically acceptable salt or ester thereof) may be administered or co-administered (e.g., consecutively, simultaneously, at different times) in any conventional dosage form. Co-administration in the context of the present disclosure refers to the use of more than one therapy in the course of a coordinated treatment to achieve an improved clinical outcome. The combination of GDNF polypeptide and SCFA or pharmaceutically acceptable salt or ester thereof described herein may be used in combination with other therapies or drugs, for example analgesics or anti-inflammatory agents.

In an embodiment, the above-mentioned treatment with a combination comprising a GDNF polypeptide and SCFA or pharmaceutically acceptable salt or ester thereof may be performed in combination with surgery (e.g., pull-through surgery of the Swenson, Soave or Duhamel type). Especially for neonates with HSCR, clinicians often recommend a trial of daily enema treatments prior to surgery. Addition of the combination of GDNF+SCFA disclosed herein to the enema might increase the likelihood that children with HSCR responded well to pre-operative enema therapy. Accordingly, in an embodiment, the above-mentioned treatment with a combination comprising a GDNF polypeptide and SCFA or pharmaceutically acceptable salt or ester thereof is performed prior to pull-through surgery. Saline enemas are also commonly used in children with HSCR after pull-through surgery. Post-surgical problems in HSCR patients are believed to be due at least in part to hypoganglionosis in retained distal bowel, the so-called “transition zone”. Thus, addition of combination comprising a GDNF polypeptide and SCFA or pharmaceutically acceptable salt or ester thereof to the enema may be useful to correct the hypoganglionosis in retained distal bowel and/or the neuronal subtype imbalance in the transition zone after surgery. Accordingly, in another embodiment, the above-mentioned treatment with a combination comprising a GDNF polypeptide and SCFA or pharmaceutically acceptable salt or ester thereof is performed after pull-through surgery.

In an embodiment, the above-mentioned treatment with a combination comprising a GDNF polypeptide and SCFA or pharmaceutically acceptable salt or ester thereof is performed in combination with ENS stem cell-based therapies, which are being considered for the treatment of HSCR. GDNF may be a useful adjunct to these therapies to promote engraftment.

HSCR is clinically subdivided into short-segment (S-HSCR) and long-segment forms (L-HSCR). S-HSCR, which occurs in >80% of cases, means the ENS is absent from rectum and sigmoid colon. L-HSCR means longer regions of distal bowel are aganglionic. The method/use described herein is for the treatment of a human patient suffering from S-HSCR or L-HSCR. In an embodiment, the method/use described herein is for the treatment of a human patient suffering from S-HSCR. In an embodiment, the method/use described herein is for the treatment of a human patient suffering from L-HSCR.

The HSCR patient may be an adult patient or pediatric patient. In an embodiment, the HSCR patient is a pediatric patient, preferably a patient that is less than 5, 4, 3 or 2 year-old, more preferably a patient that is less than 1 year-old or less than 6 month-old. In another embodiment, the patient is a male.

HSCR has been associated with mutations in RET, EDNRB, SOX10, PHOX2B, and ZFHX1B, as well as with Down syndrome or Trisomy 21 (Collagen VI-associated HSCR). There is also a significant sex difference with male to female ratio as high as 5 to 1. In an embodiment, the HSCR is Collagen VI-associated HSCR. In another embodiment, the HSCR is EDNRB mutation-associated HSCR. In an embodiment, the HSCR is male-biased HSCR. In an embodiment, the HSCR is a RET mutation-associated HSCR, e.g., HSCR associated with a mutation that reduces RET expression and/or activity in cells from the distal colon. The mutation may be a mutation in RET or in a protein involved in RET signaling. In an embodiment, the mutation is a mutation in the RET protein. Mutations in the RET gene on chromosome 10q11.2 have been shown to account for 50% of familial and 15-20% of sporadic cases of HSCR, most of which (˜75%) were associated with L-HSCR.

In another embodiment, the HSCR is not a RET mutation-associated HSCR.

### EXAMPLES

The present invention is illustrated in further details by the following non-limiting examples.

### Example 1: Materials and Methods

Mice. Homozygous Holstein^{Tg/Tg} mice (Hol^{Tg/Tg}; a model for Trisomy 21 [Collagen VI]-associated HSCR) were treated with either 10 μl enemas of GDNF (1 μg/μl), or 10 μl of GDNF combined with 5 mM of amino-butyrate acid (Butyrate) or other growth factors at 1 μg/μl, or vehicle (1×PBS) as control. Enemas were administered once a day from postnatal day (P) 4 until P8, and mice were monitored daily until P60 or megacolon-associated death. Survival graphs and statistical analyses were generated with GraphPad Prism software. One-way ANOVA followed by post-hoc Tukey multiple comparison test was carried out, and significance level was set at P<0.05. Number of biological replicates (n) of each cohort is indicated where relevant in Figures.

Example 2: Assessment of the Effect of GDNF in Combination with Neurotrophic Factors on the Survival of Hol^{Tg/Tg} Mice

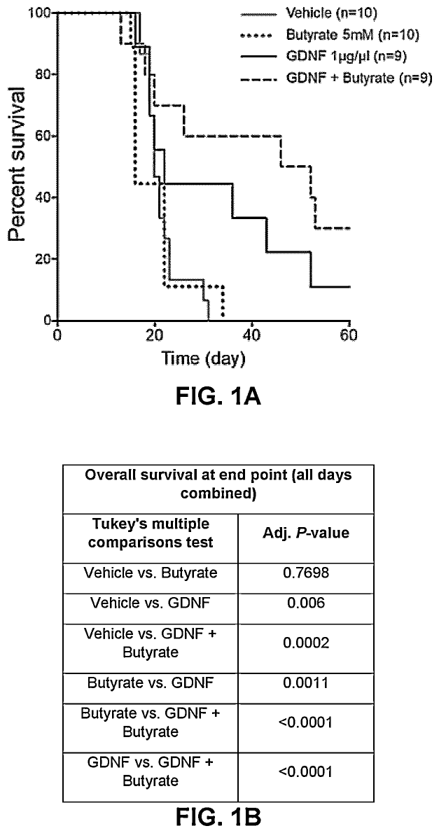

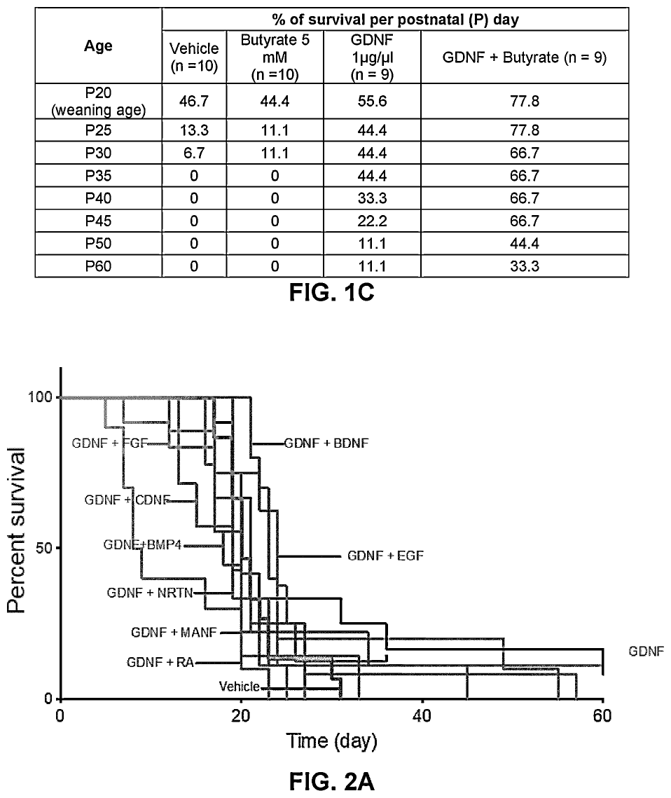

It was tested whether molecules known to have neurotrophic effects could improve the effect of GDNF on survival in a mouse model for Trisomy 21 [Collagen VI]-associated HSCR) (39). In a previous study using the same mouse model, the combination of GDNF with three molecules known to have neurotrophic effects (vitamin C, serotonin, and endothelin 3) did not improve the survival of mice relative to GDNF alone (see Soret et al., Gastroenterology. 2020 November; 159 (5): 1824-1838.e17. doi: 10.1053/j.gastro.2020.07.018. Epub 2020 Jul. 17, Supplementary FIG. 1G).

The molecules that were tested in the present study are: Bone morphogenetic protein 4 (BMP4), Nerve growth factor (NGF), Brain-derived neurotrophic factor (BDNF), Mesencephalic Astrocyte Derived Neurotrophic Factor (MANF), Cerebral Dopamine Neurotrophic Factor (CDNF), Epidermal Growth Factor (EGF), Neurturin (NRTN), Fibroblast growth factor 2 (FGF2), butyrate and Retinoic Acid (RA). These molecules have been reported to exhibit neurotrophic activity under various conditions (Table 1).

TABLE 1

| Molecule | Reported neurotrophic activity | References |
|---|---|---|
| Butyrate | Butyrate enemas enhance both cholinergic and | Soret et al, Gastroenterology |
| | nitrergic phenotype of myenteric neurons, as well as | 2010; Suply et al, Am J Physiol |
| | neuromuscular transmission in newborn rat colon. | Gastrointest Liver Physiol 2012 |
| BMP4 | BMP signaling is necessary for neural crest cell | Goldstein AM et al, Mech Dev, |
| | migration and ganglion formation in the enteric nervous | 2005; Fu M et al, Dev Biol 2006 |
| | system. | |
| NGF | NGF is chemoattractant and chemokinetic molecule for | Cornejo et al, Neurochem Res |
| | a Schwann cell precursor cell line. | 2010; Piovesana R et al, Sci |
| | | Rep 2020 |
| BDNF | Both Schwann cell- and neuron-derived BDNF are | Wilhelm et al, J Neurosci 2012; |
| | important for axon regeneration in cut peripheral | Takaki M et al, Stem Cells 2006 |
| | nerves, and BDNF induces the differentiation of | |
| | functional enteric neurons in vitro. | |
| MANF | MANF has neuroregenerative activity via differentiation | Tseng et al, eNeuro 2017; |
| | and migration of neural progenitor cells in a rat model | Lindahl M et al, Neurobiol Dis |
| | of cortical stroke. | 2017 |
| CDNF | CDNF is essential for enteric neuronal development | Lindholm et al, Nature 2007; |
| | and protects midbrain dopamine neurons in vivo. | Lindahl M et al, Neurobiol Dis |
| | | 2020; Chalazonitis A et al, J |
| | | Comp Neurol 2020 |
| EGF | EGF is chemoattractant and chemokinetic molecule for | Cornejo et al, Neurochem Res |
| | a Schwann cell precursor cell line, and enhances | 2010; Pan WK et al, |
| | proliferation and differentiation of human enteric | Neuroreport 2016 |
| | neurospheres in vitro. | |
| NRTN | Neurturin and GDNF promote the proliferation and | Heuckeroth RO et al, Dev Biol |
| | survival of enteric neuron and glial progenitors in vitro. | 1998 |
| FGF2 | FGF2 enhances the proliferation and differentiation of | Fattahi et al, Nature 2016; Pan |
| | human enteric neurospheres in vitro. | WK et al, Neuroreport 2016 |
| RA | Retinoic acid regulates murine enteric nervous system | Fattahi et al, Nature 2016; |
| | precursor proliferation and enhances neuronal | Frith T et al, Stem Cell Reports |
| | precursor differentiation. | 2020; Sato Y et al, Dev Biol |
| | | 2008 |

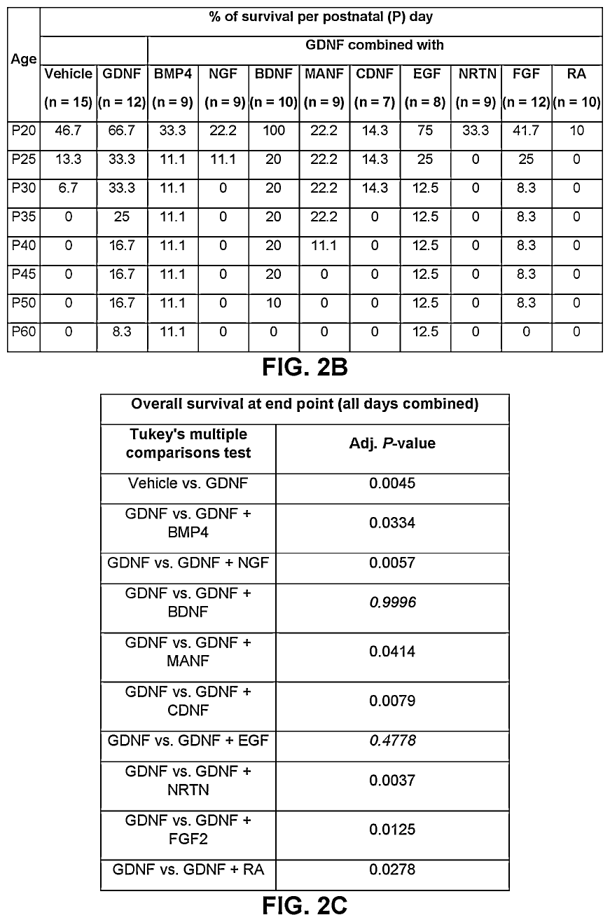

The results depicted in FIGS. 1A-C show that whereas butyrate alone has no effect on mouse survival (similar to vehicle), it significantly improves the effect of GDNF, indicating that the GDNF+butyrate combination exhibits a synergistic effect on survival. As demonstrated by the results depicted in FIGS. 2A-C, none of the other neurotrophic factors tested were able to significantly improve the effect of GDNF on survival in this mouse model. Actually, combining GDNF with some of the neurotrophic factors even reduce survival relative to administration of GDNF alone (FIG. 2C).

The results presented herein show that butyrate, but not other molecules known to exhibit neurotrophic activity, acts synergistically with GDNF to improve survival of Hol^{Tg/Tg} mice, and thus that the combination of GDNF and butyrate may be useful for the treatment of enteric neuropathies such as Hirschsprung disease in humans.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. J. B. Furness, The enteric nervous system and neurogastroenterology. Nat Rev Gastroenterol Hepatol 9, 286-294 (2012).
2. R. O. Heuckeroth, Hirschsprung disease-integrating basic science and clinical medicine to improve outcomes. Nat Rev Gastroenterol Hepatol 15, 152-167 (2018).
3. M. Rao, M. D. Gershon, Enteric nervous system development: what could possibly go wrong? Nat Rev Neurosci 19, 552-565 (2018).
4. J. Amiel et al., Hirschsprung disease, associated syndromes and genetics: a review. J Med Genet 45, 1-14 (2008).
5. R. O. Heuckeroth, K. H. Schafer, Gene-environment interactions and the enteric nervous system: Neural plasticity and Hirschsprung disease prevention. Dev Biol 417, 188-197 (2016).
6. O. Mwizerwa et al., Gdnf is mitogenic, neurotrophic, and chemoattractive to enteric neural crest cells in the embryonic colon. Dev Dyn 240, 1402-1411 (2011).
7. D. Natarajan, C. Marcos-Gutierrez, V. Pachnis, E. de Graaff, Requirement of signalling by receptor tyrosine kinase RET for the directed migration of enteric nervous system progenitor cells during mammalian embryogenesis. Development 129, 5151-5160 (2002).
8. H. M. Young et al., GDNF is a chemoattractant for enteric neural cells. Dev Biol 229, 503-516 (2001).
9. C. S. Tang et al., Identification of Genes Associated With Hirschsprung Disease, Based on Whole-Genome Sequence Analysis, and Potential Effects on Enteric Nervous System Development. Gastroenterology 155, 1908-1922 e1905 (2018).
10. E. S. Emison et al., Differential contributions of rare and common, coding and noncoding Ret mutations to multifactorial Hirschsprung disease liability. Am J Hum Genet 87, 60-74 (2010).
11. J. M. Tilghman et al., Molecular Genetic Anatomy and Risk Profile of Hirschsprung's Disease. N Engl J Med 380, 1421-1432 (2019).
12. H. Gui et al., Whole exome sequencing coupled with unbiased functional analysis reveals new Hirschsprung disease genes. Genome Biol 18, 48 (2017).
13. N. Nagy, A. M. Goldstein, Enteric nervous system development: A crest cell's journey from neural tube to colon. Semin Cell Dev Biol 66, 94-106 (2017).
14. C. S. Tang et al., Uncovering the genetic lesions underlying the most severe form of Hirschsprung disease by whole-genome sequencing. Eur J Hum Genet 26, 818-826 (2018).
15. F. Friedmacher, P. Puri, Hirschsprung's disease associated with Down syndrome: a meta-analysis of incidence, functional outcomes and mortality. Pediatric surgery international 29, 937-946 (2013).
16. O. Swenson, H. F. Rheinlander, I. Diamond, Hirschsprung's disease; a new concept of the etiology; operative results in 34 patients. N Engl J Med 241, 551-556 (1949).
17. O. Swenson, A. H. Bill, Jr., Resection of rectum and rectosigmoid with preservation of the sphincter for benign spastic lesions producing megacolon; an experimental study. Surgery 24, 212-220 (1948).
18. P. K. Tam, Hirschsprung's disease: A bridge for science and surgery. J Pediatr Surg 51, 18-22 (2016).
19. A. Coe et al., Reoperation for Hirschsprung disease: pathology of the resected problematic distal pull-through. Pediatr Dev Pathol 15, 30-38 (2012).
20. A. Pini Prato et al., Hirschsprung disease: do risk factors of poor surgical outcome exist? J Pediatr Surg 43, 612-619 (2008).
21. A. J. Burns et al., White paper on guidelines concerning enteric nervous system stem cell therapy for enteric neuropathies. Dev Biol 417, 229-251 (2016).
22. C. J. McCann, O. Borrelli, N. Thapar, Stem cell therapy in severe pediatric motility disorders. Current opinion in pharmacology 43, 145-149 (2018).
23. K. F. Bergeron, D. W. Silversides, N. Pilon, The developmental genetics of Hirschsprung's disease. Clin Genet 83, 15-22 (2013).
24. F. Obermayr, R. Hotta, H. Enomoto, H. M. Young, Development and developmental disorders of the enteric nervous system. Nat Rev Gastroenterol Hepatol 10, 43-57 (2013).
25. C. L. Yntema, W. S. Hammond, The origin of intrinsic ganglia of trunk viscera from vagal neural crest in the chick embryo. J Comp Neurol 101, 515-541 (1954).
26. N. R. Chevalier et al., How Tissue Mechanical Properties Affect Enteric Neural Crest Cell Migration. Scientific reports 6, 20927 (2016).
27. R. Hotta, R. B. Anderson, K. Kobayashi, D. F. Newgreen, H. M. Young, Effects of tissue age, presence of neurones and endothelin-3 on the ability of enteric neurone precursors to colonize recipient gut: implications for cell-based therapies. Neurogastroenterol Motil 22, 331-e386 (2010).
28. N. M. Le Douarin, M. A. Teillet, The migration of neural crest cells to the wall of the digestive tract in avian embryo. Journal of embryology and experimental morphology 30, 31-48 (1973).
29. T. Uesaka, M. Nagashimada, H. Enomoto, Neuronal Differentiation in Schwann Cell Lineage Underlies Postnatal Neurogenesis in the Enteric Nervous System. J Neurosci 35, 9879-9888 (2015).
30. Y. Watanabe et al., Morphological investigation of the enteric nervous system in Hirschsprung's disease and hypoganglionosis using whole-mount colon preparation. J Pediatr Surg 34, 445-449 (1999).
31. T. Uesaka, M. Nagashimada, H. Enomoto, GDNF signaling levels control migration and neuronal differentiation of enteric ganglion precursors. J Neurosci 33, 16372-16382 (2013).
32. S. Gianino, J. R. Grider, J. Cresswell, H. Enomoto, R. O. Heuckeroth, GDNF availability determines enteric neuron number by controlling precursor proliferation. Development 130, 2187-2198 (2003).
33. H. Wang et al., The timing and location of glial cell line-derived neurotrophic factor expression determine enteric nervous system structure and function. J Neurosci 30, 1523-1538 (2010).
34. A. Hoke et al., Glial cell line-derived neurotrophic factor alters axon schwann cell units and promotes myelination in unmyelinated nerve fibers. J Neurosci 23, 561-567 (2003).
35. G. Paratcha, F. Ledda, C. F. Ibanez, The neural cell adhesion molecule NCAM is an alternative signaling receptor for GDNF family ligands. Cell 113, 867-879 (2003).
36. D. Sjostrand, C. F. Ibanez, Insights into GFRalpha1 regulation of neural cell adhesion molecule (NCAM) function from structure-function analysis of the NCAM/GFRalpha1 receptor complex. J Biol Chem 283, 13792-13798 (2008).
37. E. Suply, P. de Vries, R. Soret, F. Cossais, M. Neunlist, Butyrate enemas enhance both cholinergic and nitrergic phenotype of myenteric neurons and neuromuscular transmission in newborn rat colon. American journal of physiology. Gastrointestinal and liver physiology 302, G1373-1380 (2012).
38. A. M. Toure, M. Landry, O. Souchkova, S. W. Kembel, N. Pilon, Gut microbiota-mediated Gene-Environment interaction in the TashT mouse model of Hirschsprung disease. Scientific reports 9, 492 (2019).
39. R. Soret et al., A collagen VI-dependent pathogenic mechanism for Hirschsprung's disease. J Clin Invest 125, 4483-4496 (2015).
40. K. F. Bergeron et al., Male-Biased Aganglionic Megacolon in the TashT Mouse Line Due to Perturbation of Silencer Elements in a Large Gene Desert of Chromosome 10. PLOS Genet 11, e1005093 (2015).
41. K. Hosoda et al., Targeted and natural (piebald-lethal) mutations of endothelin-B receptor gene produce megacolon associated with spotted coat color in mice. Cell 79, 1267-1276 (1994).
42. J. I. Lake, O. A. Tusheva, B. L. Graham, R. O. Heuckeroth, Hirschsprung-like disease is exacerbated by reduced de novo GMP synthesis. J Clin Invest 123, 4875-4887 (2013).
43. A. Schuchardt, V. D'Agati, L. Larsson-Blomberg, F. Costantini, V. Pachnis, Defects in the kidney and enteric nervous system of mice lacking the tyrosine kinase receptor Ret. Nature 367, 380-383 (1994).
44. F. Fattahi et al., Deriving human ENS lineages for cell therapy and drug discovery in Hirschsprung disease. Nature, (2016).
45. Z. Li et al., Essential roles of enteric neuronal serotonin in gastrointestinal motility and the development/survival of enteric dopaminergic neurons. J Neurosci 31, 8998-9009 (2011).
46. N. Bondurand, D. Natarajan, A. Barlow, N. Thapar, V. Pachnis, Maintenance of mammalian enteric nervous system progenitors by SOX10 and endothelin 3 signalling. Development 133, 2075-2086 (2006).
47. Z. Cheng et al., Murine model of Hirschsprung-associated enterocolitis. I: phenotypic characterization with development of a histopathologic grading system. J Pediatr Surg 45, 475-482 (2010).
48. L. S. Cheng, D. M. Schwartz, R. Hotta, H. K. Graham, A. M. Goldstein, Bowel dysfunction following pullthrough surgery is associated with an overabundance of nitrergic neurons in Hirschsprung disease. J Pediatr Surg 51, 1834-1838 (2016).
49. D. Coyle, A. M. O'Donnell, J. Gillick, P. Puri, Altered neurotransmitter expression profile in the ganglionic bowel in Hirschsprung's disease. J Pediatr Surg 51, 762-769 (2016).
50. A. M. Toure, B. Charrier, N. Pilon, Male-specific colon motility dysfunction in the TashT mouse line. Neurogastroenterol Motil 28, 1494-1507 (2016).
51. I. Zaitoun et al., Altered neuronal density and neurotransmitter expression in the ganglionated region of Ednrb null mice: implications for Hirschsprung's disease. Neurogastroenterol Motil 25, e233-244 (2013).
52. M. Luz, E. Mohr, H. C. Fibiger, GDNF-induced cerebellar toxicity: A brief review. Neurotoxicology 52, 46-56 (2016).
53. D. J. Creedon et al., Neurturin shares receptors and signal transduction pathways with glial cell line-derived neurotrophic factor in sympathetic neurons. Proc Natl Acad Sci USA 94, 7018-7023 (1997).
54. N. Pilon, D. Raiwet, R. S. Viger, D. W. Silversides, Novel pre-and post-gastrulation expression of Gata4 within cells of the inner cell mass and migratory neural crest cells. Dev Dyn 237, 1133-1143 (2008).
55. T. Shimotake, S. Go, K. Inoue, H. Tomiyama, N. Iwai, A homozygous missense mutation in the tyrosine E kinase domain of the RET proto-oncogene in an infant with total intestinal aganglionosis. Am J Gastroenterol 96, 1286-1291 (2001).
56. S. Ro, S. J. Hwang, M. Muto, W. K. Jewett, N. J. Spencer, Anatomic modifications in the enteric nervous system of piebald mice and physiological consequences to colonic motor activity. American journal of physiology. Gastrointestinal and liver physiology 290, G710-718 (2006).
57. N. M. Joseph et al., Enteric glia are multipotent in culture but primarily form glia in the adult rodent gut. J Clin Invest 121, 3398-3411 (2011).
58. G. M. Kruger et al., Neural crest stem cells persist in the adult gut but undergo changes in self-renewal, neuronal subtype potential, and factor responsiveness. Neuron 35, 657-669 (2002).
59. C. Laranjeira et al., Glial cells in the mouse enteric nervous system can undergo neurogenesis in response to injury. J Clin Invest 121, 3412-3424 (2011).
60. M. Metzger, C. Caldwell, A. J. Barlow, A. J. Burns, N. Thapar, Enteric nervous system stem cells derived from human gut mucosa for the treatment of aganglionic gut disorders. Gastroenterology 136, 2214-2225 e2211-2213 (2009).
61. M. T. Liu, Y. H. Kuan, J. Wang, R. Hen, M. D. Gershon, 5-HT4 receptor-mediated neuroprotection and neurogenesis in the enteric nervous system of adult mice. J Neurosci 29, 9683-9699 (2009).
62. K. Badizadegan et al., Presence of intramucosal neuroglial cells in normal and aganglionic human colon. American journal of physiology. Gastrointestinal and liver physiology 307, G1002-1012 (2014).
63. D. J. Wilkinson, G. S. Bethell, R. Shukla, S. E. Kenny, D. H. Edgar, Isolation of Enteric Nervous System Progenitor Cells from the Aganglionic Gut of Patients with Hirschsprung's Disease. PLOS One 10, e0125724 (2015).
64. S. Almond, R. M. Lindley, S. E. Kenny, M. G. Connell, D. H. Edgar, Characterisation and transplantation of enteric nervous system progenitor cells. Gut 56, 489-496 (2007).
65. S. Hetz et al., In vivo transplantation of neurosphere-like bodies derived from the human postnatal and adult enteric nervous system: a pilot study. PLOS One 9, e93605 (2014).
66. R. Hotta et al., Transplanted progenitors generate functional enteric neurons in the postnatal colon. J Clin Invest 123, 1182-1191 (2013).
67. J. E. Cooper et al., In Vivo Transplantation of Enteric Neural Crest Cells into Mouse Gut; Engraftment, Functional Integration and Long-Term Safety. PLOS One 11, e0147989 (2016).
68. J. E. Cooper et al., In vivo transplantation of fetal human gut-derived enteric neural crest cells. Neurogastroenterol Motil 29, (2017).
69. R. Hotta et al., Isogenic enteric neural progenitor cells can replace missing neurons and glia in mice with Hirschsprung disease. Neurogastroenterol Motil 28, 498-512 (2016).
70. L. A. Stamp et al., Optogenetic Demonstration of Functional Innervation of Mouse Colon by Neurons Derived From Transplanted Neural Cells. Gastroenterology 152, 1407-1418 (2017).
71. C. J. McCann et al., Transplantation of enteric nervous system stem cells rescues nitric oxide synthase deficient mouse colon. Nature communications 8, 15937 (2017).
72. J. Belkind-Gerson et al., Colitis promotes neuronal differentiation of Sox2+ and PLP1+ enteric cells. Scientific reports 7, 2525 (2017).
73. J. Belkind-Gerson et al., Colitis induces enteric neurogenesis through a 5-HT4-dependent mechanism. Inflamm Bowel Dis 21, 870-878 (2015).
74. M. Meir et al., Neurotrophic factor GDNF regulates intestinal barrier function in inflammatory bowel disease. J Clin Invest 129, 2824-2840 (2019).
75. D. K. Zhang et al., Glial-derived neurotrophic factor regulates intestinal epithelial barrier function and inflammation and is therapeutic for murine colitis. J Pathol 222, 213-222 (2010).
76. A. J. Burns, D. Champeval, N. M. Le Douarin, Sacral neural crest cells colonise aganglionic hindgut in vivo but fail to compensate for lack of enteric ganglia. Dev Biol 219, 30-43 (2000).
77. Y. Watanabe et al., Extrinsic nerve strands in the aganglionic segment of Hirschsprung's disease. J Pediatr Surg 33, 1233-1237 (1998).
78. H. Nakamura, T. Lim, P. Puri, Inflammatory bowel disease in patients with Hirschsprung's disease: a systematic review and meta-analysis. Pediatric surgery international 34, 149-154 (2018).
79. S. J. Mckeown, M. Mohsenipour, A. J. Bergner, H. M. Young, L. A. Stamp, Exposure to GDNF Enhances the Ability of Enteric Neural Progenitors to Generate an Enteric Nervous System. Stem Cell Reports 8, 476-488 (2017).
80. S. Srinivas et al., Cre reporter strains produced by targeted insertion of EYFP and ECFP into the ROSA26 locus. BMC Dev Biol 1, 4 (2001).
81. H. Enomoto et al., RET signaling is essential for migration, axonal growth and axon guidance of developing sympathetic neurons. Development 128, 3963-3974 (2001).
82. S. Jain et al., Mice expressing a dominant-negative Ret mutation phenocopy human Hirschsprung disease and delineate a direct role of Ret in spermatogenesis. Development 131, 5503-5513 (2004).
83. A. Boulende Sab et al., An Ebox element in the proximal Gata4 promoter is required for Gata4 expression in vivo. PLOS ONE 6, e29038 (2011).
84. I. Laforest-Lapointe, A. Paquette, C. Messier, S. W. Kembel, Leaf bacterial diversity mediates plant diversity and ecosystem function relationships. Nature 546, 145-147 (2017).
85. P. D. Schloss et al., Introducing mothur: open-source, platform-independent, community-supported software for describing and comparing microbial communities. Appl Environ Microbiol 75, 7537-7541 (2009).
86. D. McDonald et al., The Biological Observation Matrix (BIOM) format or: how I learned to stop worrying and love the ome-ome. Gigascience 1, 7 (2012).
87. R. C. Team, R: A language and environment for statistical computing. R Foundation for Statistical Computing, Vienna, Austria. (2014).

1. A method for treating a human subject suffering from an enteric neuropathy, the method comprising administrating to the subject an effective amount of (i) a Glial cell line-Derived Neurotrophic Factor (GDNF) polypeptide; and (ii) a short chain fatty acid (SCFA), or a pharmaceutically acceptable salt or ester thereof.

2. The method of claim 1, wherein the GDNF polypeptide comprises an amino acid sequence having at least 70% identity with amino acids 78-211 of SEQ ID NO: 1.

3. (canceled)

4. The method of claim 2, wherein the GDNF polypeptide comprises an amino acid sequence having at least 95% identity with amino acids 78-211 of SEQ ID NO:1.

5. The method of claim 4, wherein the GDNF polypeptide comprises amino acids 78-211 of SEQ ID NO:1.

6. (canceled)

7. The method of claim 1, wherein the SCFA is a C₃-C₅ SCFA.

8. The method of claim 1, wherein the SCFA is butyric acid.

9. The method of claim 1, wherein the GDNF polypeptide and/or the SCFA or pharmaceutically acceptable salt or ester thereof are present in a pharmaceutical composition that further comprises a pharmaceutically acceptable carrier.

10. The method of claim 9, wherein the pharmaceutically acceptable carrier comprises a saline solution or a gelling agent.

11. The method of claim 1, wherein the GDNF polypeptide and/or the SCFA or pharmaceutically acceptable salt or ester thereof are administered rectally through enema.

12. The method of claim 1, wherein the GDNF polypeptide and/or the SCFA or pharmaceutically acceptable salt or ester thereof are administered by injection into the distal colon wall.

13-14. (canceled)

15. The method of claim 1, wherein the method is performed prior to and/or after surgical removal of the aganglionic or hypoganglionic segment in the subject.

16. (canceled)

17. The method of claim 15, wherein the surgical removal of the aganglionic or hypoganglionic segment is through pull-through surgery.

18. The method of claim 1, wherein the enteric neuropathy is intestinal hypoganglionosis.

19. The method of claim 1, wherein the enteric neuropathy is Hirschsprung disease (HSCR).

20. The method of claim 19, wherein the subject suffers from short-segment HSCR.

21. The method of claim 19, wherein the HSCR is Collagen VI-associated HSCR.

22. The method of claim 1, wherein the human subject is less than 5-year-old.

23. (canceled)

24. The method of claim 1, wherein the GDNF polypeptide and/or the SCFA or pharmaceutically acceptable salt or ester thereof are administered into the rectum and/or the sigmoid colon.

25. The method of claim 24, wherein the GDNF polypeptide and/or the SCFA or pharmaceutically acceptable salt or ester thereof are administered into the rectosigmoid region.

26. A combination comprising (i) a GDNF polypeptide; and (ii) an SCFA, or a pharmaceutically acceptable salt or ester thereof.

27-50. (canceled)
